(19) United States
(12) Patent Application Publication
(10) Pub. No.: US 2020/0221730 A1
(43) Pub. Date: Jul. 16, 2020

(54) INACTIVATION OF AFRICAN SWINE FEVER VIRUS USING A FEED ADDITIVE

(71) Applicant: KEMIN INDUSTRIES, INC., Des Moines, IA (US)

(72) Inventor: Megan Niederwerder, Manhattan, KS (US)

(21) Appl. No.: 16/580,926

(22) Filed: Sep. 24, 2019

Related U.S. Application Data
(60) Provisional application No. 62/792,552, filed on Jan. 15, 2019.

(51) Int. Cl. A23K 20/105; A23K 10/16; A61K 47/12; A01K 67/02

(52) U.S. Cl. CPC A23K 20/105; A23K 10/16; A23K 50/30; A01K 67/02; A61K 47/12

(57) ABSTRACT

African swine fever virus (ASFV) is a very large complex DNA virus that is rapidly spreading through the largest pork producing country in the world, China. ASFV causes high mortality in pigs and is currently a foreign animal disease to North America and most European countries. There is currently no effective vaccine and the virus is known to be transmitted through the oral route via consumption of contaminated feed. ASFV is capable of surviving in feed and feed ingredients subjected to varying environmental conditions simulating transoceanic shipment. The present invention relates to a feed additive that is effective at mitigating ASFV in cell culture and in feed and feed ingredients.

### CROSS REFERENCE TO RELATED APPLICATIONS

The present application claims the benefit of priority to U.S. Provisional Patent Application No. 62/792,552, filed Jan. 15, 2019, entitled “INACTIVATION OF AFRICAN SWINE FEVER VIRUS USING A FEED ADDITIVE,” which is hereby incorporated by reference in its entirety.

### SUMMARY OF THE INVENTION

African Swine Fever Virus (ASFV) is a very large complex DNA virus that is rapidly spreading through the largest pork producing country in the world, China. ASFV causes high mortality in pigs and is currently a foreign animal disease to North America and most European countries. There is currently no effective vaccine and the virus is known to be transmitted through the oral route via consumption of contaminated feed. ASFV is capable of surviving in feed and feed ingredients subjected to varying environmental conditions simulating transoceanic shipment. At least one aspect of the present invention relates to a feed additive that is effective at mitigating ASFV in cell culture and in feed and feed ingredients.

Another aspect of the present invention relates to combining or administering a mitigant against ASFV to animal feed or feed ingredients, wherein the mitigant is a composition that contains an effective amount of aqueous formaldehyde and proprionic acid. For instance, at least one embodiment of the present invention relates to the use of SalCURB™, a feed additive that contains aqueous formaldehyde and propionic acid, Kemin Industries, Inc. (Des Moines, Iowa), as a mitigant against ASFV. It is commercially available and labeled to control Salmonella in complete feeds and feed ingredients. Safety testing has been performed on formaldehyde, and formaldehyde (as used in SalCURB) is FDA-approved for application on livestock and poultry feeds and feed ingredients at a rate of 6.5 lbs per ton (0.33%). When applied at the 6.5 lbs per ton recommended rate, SalCURB provides the equivalent of 37% aqueous formaldehyde at the rate of 5.4 pounds per ton of feed. See 21 CFR 573.460. It is not currently labeled for control of any viruses. However, experimental evidence has demonstrated efficacy against porcine epidemic diarrhea virus (PEDV). See U.S. Published Patent Application No. 2017/0354167A1, which is incorporated in its entirety herein. Although SalCURB has been shown to be effective against porcine epidemic diarrhea virus (PEDV) and Salmonella sp., the researcher's data reports shows SalCURB has been shown to be an effective mitigant against ASFV, a disease foreign to the United States industry. ASFV is a very unique virus and is the only virus in the family Asfarviridae and genus Asfivirus. Importantly, there are no appropriate surrogate viruses for ASFV. Thus, the effects of mitigants on other viruses or bacteria cannot be extended or translated to ASFV without direct evidence of the mitigant on the virus itself. To the researcher's knowledge, this constitutes the earliest available data showing that SalCURB inclusion rates of 0.33% (the FDA approved inclusion rate) results in inactivation of ASFV in feed. Additionally, the researcher has developed a cell culture experimental system to determine the level at which SalCURB is an effective mitigant for inactivation of ASFV. The standard inclusion of SalCURB is 0.33%; however, a laboratory culture model has demonstrated that concentrations of SalCURB as low as 0.0625% significantly reduce the viral titer by approximately 1.4 logs. The researcher's work has also demonstrated that SalCURB effectively inactivates ASFV when used in feed and feed ingredients during simulated transboundary shipment. The data demonstrates that SalCURB significantly reduces the quantity of ASFV DNA within as little as 1 day after exposure in most of the ingredients tested. Additionally, all samples treated with SalCURB were negative when tested on virus isolation and bioassay. To the researcher's knowledge, this is the first work demonstrating that a commercially available feed additive SalCURB is an effective mitigant of ASFV in cell culture, feed and feed ingredients. At the time of filing, there are no currently available commercial products approved by the FDA for mitigation of ASFV in feed.

### BRIEF DESCRIPTION OF THE DRAWINGS

FIG. 1 depicts the dose response inactivation curve of ASFV (strain BA71V) exposed to varying concentrations of SalCURB. Data is shown as the ASFV titer after exposure to SalCURB concentrations between 0.03125% and 2.0% (grey bars) along with the percent reduction of virus concentration (line) compared to the positive control (black bar). SalCURB exposure occurred for 30 minutes at room temperature prior to virus plating on cell culture.

FIG. 2 depicts the detection of ASFV Georgia 2007 DNA over the course of the 30 day transboundary model. Data is shown as the mean cycle threshold (Ct) values for duplicate replicates at days 1, 8, 17 and 30 post-inoculation. Data is shown for untreated controls (open boxes) and samples treated with 0.33% SalCURB immediately prior to ASFV-inoculation on 0 dpi (black boxes). All samples had detectable ASFV DNA at the conclusion of the 30 day transboundary model. However, SalCURB significantly increased the Ct values of treated samples, indicating a reduction in viral DNA compared to the positive controls. Ct values >40 were considered negative.

FIG. 3 depicts the quantity of ASFV Georgia 2007 DNA as measured by qPCR at the conclusion of the 30 day transboundary model in nontreated controls (open bars) and samples treated with 0.33% SalCURB at 28 dpi (black bars). Data is shown as the mean cycle threshold (Ct) of duplicate replicates. All samples were positive for ASFV DNA at the conclusion of the transboundary model. However, SalCURB significantly increased the Ct values of treated soybean meal conventional and organic samples, indicating a reduction in viral DNA compared to the positive controls. Ct values >40 were considered negative.

### DETAILED DESCRIPTION OF THE INVENTION

At least one aspect of the present invention relates to a feed additive that is effective at mitigating ASFV in cell culture and in feed and feed ingredients. Another aspect of the present invention relates to combining or administering a mitigant against ASFV to animal feed or feed ingredients, wherein the mitigant is a composition that contains an effective amount of aqueous formaldehyde and proprionic acid. For instance, at least one embodiment of the present invention relates to the use of SalCURB™ as a mitigant against ASFV.

According to at least one embodiment, the aqueous formaldehyde is a solution comprising between about 20% to about 54% aqueous formaldehyde, more preferably, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54% aqueous formaldehyde. For instance, in at least one embodiment, the aqueous formaldehyde solution comprises between about 28% to about 46% aqueous formaldehyde, such as 37% aqueous formaldehyde.

According to at least one embodiment, the propionic acid is present in an amount from about 0.00001% to about 15% by weight of the blend. For instance, in at least one embodiment, the propionic acid is present in an amount of at least 0.03%. In at least one embodiment, the propionic acid is present in an amount ranging between about 0.03% to about 10%, such as an amount ranging between about 0.0625% to about 0.33%.

The present invention further relates to a method of controlling African Swine Fever Virus (ASFV) comprising the step of combining a composition comprising an effective amount of aqueous formaldehyde and propionic acid with a food product and/or animal feed and/or a component of a food product and/or animal feed. According to at least one embodiment, the food product and/or animal feed and/or component of a food product and/or animal feed is in a processed food chain for consumption by an animal selected from the group consisting of a mammal or a bird. According to at least one embodiment, the animal is in the Sus genus. In at least one embodiment, the food product and/or animal feed and/or component of a food product and/or animal feed in a processed food chain is selected from the group consisting of raw feed materials and processed food.

According to at least one embodiment, the mitigant is applied to high-risk feed ingredients, including but not limited to include soybean meal conventional, soybean meal organic, soy oilcake, choline, moist cat food, moist dog food, dry dog food, pork sausage casings, and complete feed.

According to at least one embodiment, the composition comprising aqueous formaldehyde and propionic acid further comprises a quantity of at least one ingredient selected from the group consisting of methanol, water, sodium hydroxide, mono glycerides, diglycerides, and any combination thereof.

Another aspect of the present invention relates to controlling of ASFV includes the prevention of transmission of active ASFV to an animal consuming the food product and/or a component of a food product in a processed food chain. In another aspect, the controlling of ASFV includes the inactivation of ASFV in the food product and/or animal feed and/or a component of a food product and/or animal feed. In at least one embodiment, the controlling of ASFV includes a decrease in the presence of active ASFV by at least 1 log after the composition is combined with the food product and/or animal feed and/or a component of a food product and/or animal feed.

### Examples

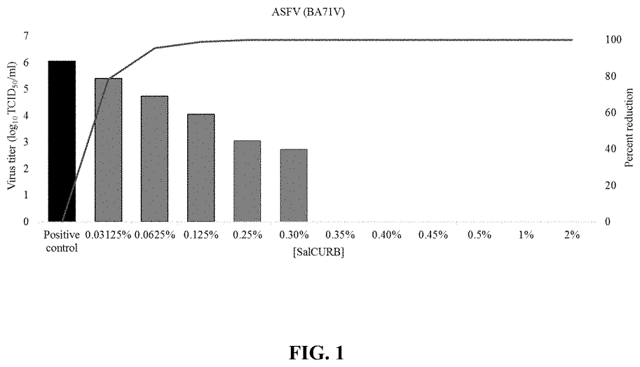

The researcher developed protocols and procedures for diluting and mixing various concentrations of SalCURB with ASFV (BA71v isolate) in vero cells. As a first step, the SalCURB was prepared at concentrations ranging between 2% and 0.03125% and mixed with a standard high concentration of ASFV (10⁶ TCID₅₀/ml). The researcher included positive controls in each assay to determine the dose response inactivation of the virus. Results (Tables 1-3, FIG. 1) demonstrated that SalCURB effectively inactivated ASFV to undetectable levels by indirect fluorescent antibody testing at all doses tested between 0.35% and 2.0%. Dose dependent reduction of ASFV was seen at SalCURB concentrations between 0.03125% and 0.3%. At the lowest concentration of SalCURB tested in cell culture (0.03125%), there was an approximate 0.7 log₁₀ TCID₅₀/ml reduction in virus titer. At 0.0625% SalCURB inclusion, virus titers were reduced by approximately 95.3%. An approximate 3.4 log reduction in virus titer was seen at 0.3% SalCURB inclusion. A 4 log reduction in virus titer is the standard described by the World Organization for Animal Health (OIE) for virus inactivation. In summary, the researcher demonstrated that SalCURB is an effective inactivant of ASFV in cell culture and that the dose required for an approximate 4 log reduction is 0.3%, a dose lower than the standard 0.33% inclusion rate.

TABLE 1
Inactivation of ASFV (BA71V isolate) at varying concentrations of SalCURB
after 30 minutes of incubation at room temperature

| | 2% | | | 1% | | | 0.5% | | | 0.25% | | | 0.125% | | | Pos. ctrl | | |
|---|---|---|---|---|---|---|---|---|---|---|---|---|---|---|---|---|---|---|
| undil. | − | − | − | − | − | − | − | − | − | − | − | − | − | − | − | + | + | + |
| 10⁻¹ | − | − | − | − | − | − | − | − | − | − | − | − | − | − | − | + | + | + |
| 10⁻² | − | − | − | − | − | − | − | − | − | − | + | + | + | + | + | + | + | + |
| 10⁻³ | − | − | − | − | − | − | − | − | − | − | − | − | − | + | + | + | + | + |
| 10⁻⁴ | − | − | − | − | − | − | − | − | − | − | − | − | − | − | − | + | + | + |
| 10⁻⁵ | − | − | − | − | − | − | − | − | − | − | − | − | − | − | − | − | + | + |
| 10⁻⁶ | − | − | − | − | − | − | − | − | − | − | − | − | − | − | − | − | − | − |
| 10⁻⁷ | − | − | − | − | − | − | − | − | − | − | − | − | − | − | − | − | − | − |

| | | | | | | |
|---|---|---|---|---|---|---|
| TCID₅₀/ml | − | − | − | 1.17 × 10³ = 10^{3.1} | 1.17 × 10⁴ = 10^{4.1} | 1.17 × 10⁶ = 10^{6.1} |
| Log | − | − | − | 3 | 2 | − |
| decrease | | | | | | |

*Data is shown as positive (+) or negative (−) for ASFV on virus isolation. Virus titration was performed on vero cells in triplicate using a monoclonal Ab against ASFV p30.

TABLE 2
Inactivation of ASFV (BA71V isolate) at varying concentrations of SalCURB
after 30 minutes of incubation at room temperature

| | 0.45% | | | 0.4% | | | 0.35% | | | 0.3% | | | 0.25% | | | Pos. ctrl | | |
|---|---|---|---|---|---|---|---|---|---|---|---|---|---|---|---|---|---|---|
| undil. | − | − | − | − | − | − | − | − | − | − | − | − | − | − | − | + | + | + |
| 10⁻¹ | − | − | − | − | − | − | − | − | − | − | − | − | − | − | − | + | + | + |
| 10⁻² | − | − | − | − | − | − | − | − | − | − | − | + | − | + | + | + | + | + |
| 10⁻³ | − | − | − | − | − | − | − | − | − | − | − | − | − | − | − | + | + | + |
| 10⁻⁴ | − | − | − | − | − | − | − | − | − | − | − | − | − | − | − | + | + | + |
| 10⁻⁵ | − | − | − | − | − | − | − | − | − | − | − | − | − | − | − | + | − | + |
| 10⁻⁶ | − | − | − | − | − | − | − | − | − | − | − | − | − | − | − | − | − | − |
| 10⁻⁷ | − | − | − | − | − | − | − | − | − | − | − | − | − | − | − | − | − | − |

| | | | | | | |
|---|---|---|---|---|---|---|
| TCID₅₀/ml | − | − | − | 5.45 × 10² = 10^{2.7} | 1.17 × 10³ = 10^{3.1} | 1.17 × 10⁶ = 10^{6.1} |
| Log | − | − | − | 3.4 | 3 | − |
| decrease | | | | | | |

*Data is shown as positive (+) or negative (−) for ASFV on virus isolation. Virus titration was performed on vero cells in triplicate using a monoclonal Ab against ASFV p30.

TABLE 3
Inactivation of ASFV (BA71V isolate) at varying concentrations of SalCURB
after 30 minutes of incubation at room temperature

| | 0.125% | | | 0.0625% | | | 0.03125% | | | Pos. ctrl | | |
|---|---|---|---|---|---|---|---|---|---|---|---|---|
| undil. | − | − | − | − | − | − | − | − | − | + | + | + |
| 10⁻¹ | − | − | − | − | − | − | − | − | − | + | + | + |
| 10⁻² | + | + | + | + | + | + | + | + | + | + | + | + |
| 10⁻³ | − | + | + | + | + | + | + | + | + | + | + | + |
| 10⁻⁴ | − | − | − | − | − | + | + | + | + | + | + | + |
| 10⁻⁵ | − | − | − | − | − | − | − | − | − | − | + | + |
| 10⁻⁶ | − | − | − | − | − | − | − | − | − | − | − | − |
| 10⁻⁷ | − | − | − | − | − | − | − | − | − | − | − | − |

| | | | | |
|---|---|---|---|---|
| TCID₅₀/ml | 1.17 × 10⁴ = 10^{4.1} | 5.45 × 10⁴ = 10^{4.7} | 2.53 × 10⁵ = 10^{5.4} | 1.17 × 10⁶ = 10^{6.1} |
| Log | 2 | 1.4 | 0.7 | − |
| decrease | | | | |

*Data is shown as positive (+) or negative (−) for ASFV on virus isolation. Virus titration was performed on vero cells in triplicate using a monoclonal Ab against ASFV p30.

TABLE 4
Detection of ASFV Georgia 2007 by virus isolation at the conclusion
of the 30 day transboundary model in feed and feed ingredients
exposed to SalCURB at 0 days post-inoculation (dpi) or 28 dpi*

| | | No SalCURB | SalCURB | SalCURB |
|---|---|---|---|---|
| Sample | Feed Ingredients | Treatment† | at 0 dpi | at 28 dpi |
| 1 | Soybean meal - | + (10^{3.0}) | − | − |
| | Conventional | | | |
| 2 | Soybean meal - | + (10^{3.0}) | − | − |
| | Organic | | | |
| 3 | Soy oilcake | + (10^{3.1}) | − | − |
| 6 | Choline | + (10^{3.2}) | − | − |
| 8 | Moist cat food | + (10^{3.0}) | − | − |
| 9 | Moist dog food | + (10^{2.8}) | − | − |
| 10 | Dry dog food | + (10^{2.7}) | − | − |
| 11 | Pork sausage casings | + (10^{2.9}) | − | − |
| 12 | Positive control | + (10^{2.7}) | − | − |
| | complete feed | | | |
| 13 | Negative control | − | ND | ND |
| | complete feed | | | |

*Data is shown as positive (+) or negative (−) for ASFV on virus isolation at 30 dpi. Virus isolation was performed on porcine alveolar macrophages in triplicate using a monoclonal Ab against ASFV p30.
ND, not determined.
†Titers are shown as mean TCID₅₀ in positive controls; Initial virus inoculation was 10⁵ TCID₅₀

TABLE 5
Detection of ASFV Georgia 2007 by pig bioassay at the conclusion
of the 30 day transboundary model in feed and feed ingredients
exposed to SalCURB at 0 days post-inoculation (dpi) or 28 dpi*

| Pig | Pooled | SalCURB | Pig | Pooled | SalCURB |
|---|---|---|---|---|---|
| Number | Samples† | at 0 dpi | Number | Samples† | at 28 dpi |
| 058 | 11 | − | 229 | 9 | − |
| 057 | 1, 8 | − | 230 | 8, 11 | − |
| 059 | 2, 12 | − | 228 | 10, 12 | − |
| 055 | 3, 6 | − | 231 | 1, 2 | − |
| 060 | 9, 10 | − | 227 | 3, 6 | − |
| 050 | 13 | − | 232 | 13 | − |

*Data is shown as positive (+) or negative (−) bioassay results for ASFV after intramuscular injection of feed supernatant collected at 30 dpi and tested in nursery pigs. No more than 2 samples were tested in each pig. Samples were pooled based on PCR values from 30 dpi. Positive and negative results were determined based on ASFV PCR of serum and spleen, and virus isolation on spleen from inoculated pigs. Samples were considered positive for the presence of infectious ASFV if one or more of the diagnostic tests were positive.
†Key: 1, Soybean meal - Conventional; 2, Soybean meal - Organic; 3, Soy oilcake; 6, Choline; 8, Moist cat food; 9, Moist dog food; 10, Dry dog food; 11, Pork sausage casings; 12, Positive control complete feed; 13, Negative control complete feed

The researcher also tested a 0.33% SalCURB inclusion rate in 9 high-risk ingredients for ASFV survival using the ASFV Georgia 2007 isolate in a 30 transboundary model that simulates varying environmental temperature and humidity conditions. ASFV Georgia 2007 is the highly virulent ASFV isolate currently circulating in China and Europe. The high-risk feed ingredients were based on previous work (Dee et al., 2018) and include soybean meal conventional, soybean meal organic, soy oilcake, choline, moist cat food, moist dog food, dry dog food, pork sausage casings, and complete feed. Detection and quantification of ASFV DNA was performed by qPCR and compared between untreated inoculated feed and SalCURB-treated inoculated feed.

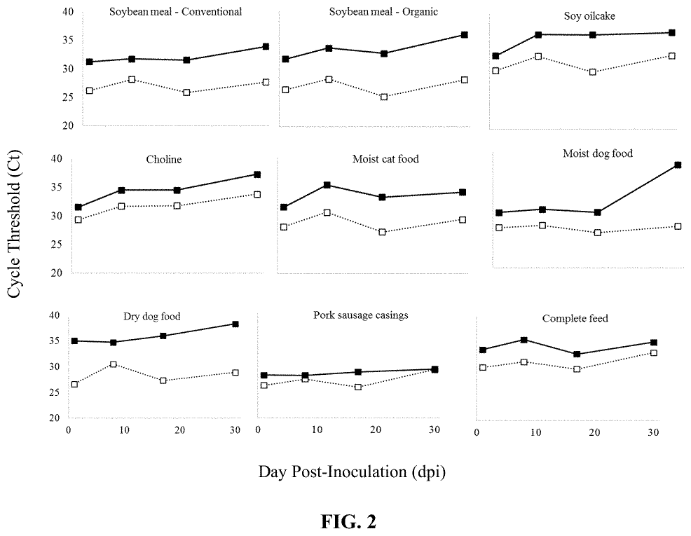

In the first study, feed was treated with 0.33% SalCURB inclusion at 0 days post-inoculation (dpi) immediately prior to ASFV inoculation. The PCR results demonstrated that all untreated control samples and 0 dpi SalCURB-treated samples were positive for ASFV DNA on days 1, 8, 17 and 30 (FIG. 2). However, in almost all feed ingredients, treatment with SalCURB resulted in a significant reduction of ASFV DNA present in the sample. This was true even as early as 1 dpi in most feed ingredients. The feed ingredients where SalCURB treatment resulted in consistent reductions of ASFV DNA were soybean meal conventional and organic, soy oilcake, choline, moist cat and dog food, dry dog food and complete feed. The only ingredient in which SalCURB did not significantly reduce ASFV DNA at all time points was pork sausage casings (FIG. 2).

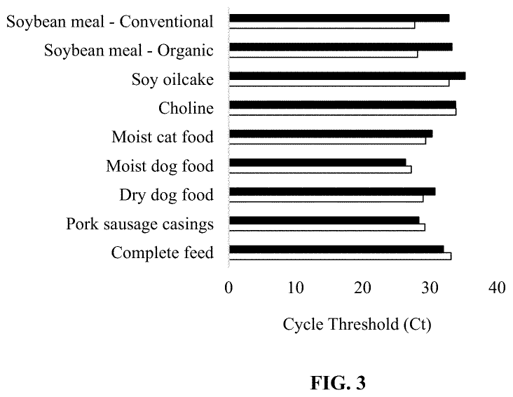

In the second study, detection and quantification of ASFV DNA was compared between untreated inoculated feed and inoculated feed treated with SalCURB at 28 dpi. The results demonstrated that all untreated and 28 dpi SalCURB-treated samples were positive for ASFV DNA at 30 dpi. However, significant reductions in ASFV DNA were noted most prominently by an increase of 5 Ct (Cycle threshold) in soybean meal conventional and organic after only 2 days of SalCURB treatment (FIG. 3).

Untreated inoculated feed ingredients and inoculated feed ingredients treated with 0.33% SalCURB at both 0 dpi and 28 dpi were then tested by virus isolation on porcine alveolar macrophages to determine if the ASFV DNA detected by PCR at 30 dpi was infectious on cell culture. Virus isolation determined that infectious virus was present in all untreated positive controls at approximate titers of 10³ TCID₅₀, whereas infectious virus was not detectable in any samples treated with SalCURB at either 0 dpi or 28 dpi (Table 3). Infectious virus was detected in positive untreated samples using a monoclonal antibody against the ASFV p30 protein.

Feed and feed ingredients treated with SalCURB at either 0 dpi or 28 dpi were then further tested in a nursery pig bioassay model to assess for the presence of infectious virus. Supernatant samples from SalCURB-treated feed at 30 dpi were injected intramuscularly as this is the most sensitive method to detect infectious ASFV. Pigs were injected with either 1 or 2 samples to reduce the number of pigs utilized. Pooled samples were based on quantitative PCR results. All feed samples treated with SalCURB at 0 dpi and 28 dpi were negative for infectious ASFV on pig bioassay (Tables 4, 5).

Overall, the data supports SalCURB being an effective mitigant for infectious ASFV in cell culture and in feed ingredients. This is the first and to the researcher's knowledge, the only, data which shows SalCURB could be used as a feed additive to inactivate ASFV in feed and feed ingredients. With recent reports of ASFV contaminated feed ingredients being detected in China, a label claim for SalCURB being effective against ASFV would be extremely valuable to the feed industry. The researcher has demonstrated the efficacy of SalCURB on ASFV through multiple mechanisms, including 1) a cell culture model and dose response inactivation curve demonstrating the necessary inclusion rate, 2) PCR quantification of ASFV DNA in SalCURB treated feed and feed ingredients, 3) virus isolation of feed and feed ingredient samples treated with SalCURB, and 4) pig bioassay of feed and feed ingredients treated with SalCURB. These all demonstrate that SalCURB is an effective and promising mitigant against ASFV in feed and feed ingredients.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

Cochrane, et al. 2016. Evaluating Chemical Mitigation of Salmonella Typhimurium ATCC 14028 in Animal Feed Ingredients. J Food Prot. 79(4): 672-6.
Dee, et al. 2016. Modeling the transboundary risk of feed ingredients contaminated with porcine epidemic diarrhea virus. BMC Vet Res. 12:51.
Dee, et al. 2015. An evaluation of porcine epidemic diarrhea virus survival in individual feed ingredients in the presence or absence of a liquid antimicrobial. Porcine Health Manager 1:9.

What is claimed is:

1. A method of controlling African Swine Fever Virus (ASFV) comprising the step of combining a composition comprising an effective amount of aqueous formaldehyde and propionic acid with a food product and/or animal feed and/or a component of a food product and/or animal feed.

2. The method of claim 1, wherein the food product and/or animal feed and/or component of a food product and/or animal feed is in a processed food chain for consumption by an animal selected from the group consisting of a mammal or a bird.

3. The method of claim 1, wherein the animal is in the Sus genus.

4. The method of claim 1, wherein the food product and/or animal feed and/or component of a food product and/or animal feed in a processed food chain is selected from the group consisting of raw feed materials and processed food.

5. The method of claim 1, wherein the aqueous formaldehyde is a solution comprising between about 20% to about 54% aqueous formaldehyde, more preferably, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54% aqueous formaldehyde.

6. The method of claim 5, wherein the aqueous formaldehyde solution comprises between about 28% to about 46% aqueous formaldehyde.

7. The method of claim 5, wherein the aqueous formaldehyde solution comprises about 37% aqueous formaldehyde.

8. The method of claim 1, wherein the propionic acid is present in an amount from about 0.00001% to about 15% by weight of the blend.

9. The method of claim 1, wherein the amount of aqueous formaldehyde and propionic acid is within +/−10% of the aqueous formaldehyde and propionic acid content of SalCURB™ (KEMIN, Des Moines, Iowa).

10. The method of claim 9, wherein the amount of aqueous formaldehyde and propionic acid is identical to the aqueous formaldehyde and propionic acid content of SalCURB™.

11. The method of claim 1, wherein the composition comprising aqueous formaldehyde and propionic acid further comprises a quantity of at least one ingredient selected from the group consisting of methanol, water, sodium hydroxide, mono glycerides, diglycerides, and any combination thereof.

12. The method of claim 1, wherein the controlling of ASFV includes the prevention of transmission of active ASFV to an animal consuming the food product and/or a component of a food product in a processed food chain.

13. The method of claim 1, wherein the controlling of ASFV includes the inactivation of ASFV in the food product and/or animal feed and/or a component of a food product and/or animal feed.

14. The method of claim 1, wherein the controlling of ASFV includes a decrease in the presence of active ASFV by at least 1 log after the composition is combined with the food product and/or animal feed and/or a component of a food product and/or animal feed.

15. The method of claim 1, wherein the composition contains aqueous formaldehyde and propionic acid in an amount of at least 0.03%.

16. The method of claim 1, wherein the composition contains aqueous formaldehyde and propionic acid in an amount ranging between about 0.03% to about 10%.

17. The method of claim 1, wherein the composition contains aqueous formaldehyde and propionic acid in an amount ranging between about 0.0625% to about 0.33%.
